(19) United States
(12) Patent Application Publication
(10) Pub. No.: US 2011/0124971 A1
(43) Pub. Date: May 26, 2011

(54) PORTAL ASSEMBLY WITH MULTI-SEAL SYSTEM

(75) Inventors: Jose A. Ramos, Ponce, PR (US); Lemuel Guisao, Ponce, PR (US)

(73) Assignee: Tyco Healthcare Group LP

(21) Appl. No.: 12/939,225

(22) Filed: Nov. 4, 2010

Related U.S. Application Data
(60) Provisional application No. 61/263,059, filed on Nov. 20, 2009.

(51) Int. Cl. A61B 1/32

(52) U.S. Cl. 600/208

(57) ABSTRACT

A surgical portal assembly is provided and includes a portal housing and a portal member mounted to the portal housing. The portal member dimensioned for positioning within tissue to access an underlying surgical site. The portal housing and portal member define a central longitudinal axis and a longitudinal passageway for passage of a surgical object. A seal mount is mounted to the portal housing and includes first and second seal members. Each of the first and second seal members has inner surfaces defining a passage for reception of the surgical object in substantial sealed reception. The first seal member adapted for angulating movement relative to the central longitudinal axis upon angulation of the surgical instrument while substantially maintaining the sealed reception of the surgical object. The second seal member being generally fixed from angulating movement.

### CROSS-REFERENCE TO RELATED APPLICATIONS

This application claims priority to and the benefit of U.S. Provisional Application Ser. No. 61/263,059 filed on Nov. 20, 2009, the entire contents of which are incorporated herein by reference.

### BACKGROUND

1. Technical Field

The present disclosure relates to a surgical portal apparatus for accessing an underlying body cavity, and, in particular, relates to a multi-seal system for use with an introducer which is intended for insertion into a patient's body, and to receive an instrument in sealing engagement therewith.

2. Description of the Related Art

Minimally invasive and laparoscopic procedures generally require that any instrumentation inserted into the body is sealed, i.e., provisions must be made to ensure that gases and/or fluids do not enter or exit the body through an endoscopic incision, such as, for example in surgical procedures where the surgical region is insufflated. For such procedures, the introduction of a tube into anatomical cavities, such as the peritoneal cavity, is usually accomplished by use of a system incorporating a trocar and cannula assembly. Since the cannula is in direct communication with the interior of the peritoneal cavity, insertion of the cannula into an opening in the patient's body to reach the inner abdominal cavity should be adapted to maintain a fluid tight interface between the abdominal cavity and the outside atmosphere. In view of the need to maintain the atmospheric integrity of the inner area of the cavity, a seal assembly for a cannula, which permits introduction of a wide range of surgical instrumentation and maintains the atmospheric integrity of the inner area of the cavity is desirable. In this regard, there have been a number of attempts in the prior art to achieve such sealing requirements. A difficulty encountered with conventional seal assemblies, however, is the inability of accommodating the wide range of sizes of instrumentation. In addition, angulation and/or manipulation of instrumentation within the cannula often present difficulties with respect to maintaining seal integrity.

### SUMMARY

Accordingly, the present disclosure provides a surgical portal assembly. The surgical port includes a portal housing and a portal member mounted to the portal housing. The portal member dimensioned for positioning within tissue to access an underlying surgical site. The portal housing and portal member define a central longitudinal axis and a longitudinal passageway for passage of a surgical object. A seal mount is mounted to the portal housing and includes first and second seal members. Each of the first and second seal members has inner surfaces defining a passage for reception of the surgical object in substantial sealed reception. The first seal member adapted for angulating movement relative to the central longitudinal axis upon angulation of the surgical instrument while substantially maintaining the sealed reception of the surgical object. The second seal member being generally fixed from angulating movement.

In an embodiment, the seal mount includes a third seal member. The third seal member is generally fixed from angulating movement. The first seal member may be disposed between the second and third seal members. The second seal member may be in contacting relation with at least one of the first and third seal members

In certain embodiments, each of the first, second and third seal members define a generally spherical segment.

In an embodiment, the inner surfaces of the first seal member define an open aperture in the absence of the surgical object.

In an embodiment, the first seal member is normally biased to an aligned position where the seal passage of the first seal member is in general longitudinal alignment with the central longitudinal axis. The first seal member may include resilient biasing segments extending in a general proximal direction and dimensioned to operatively engage the portal housing. The biasing segments adapted to bias the seal passage to the aligned position.

### BRIEF DESCRIPTION OF THE DRAWINGS

The foregoing features of the present disclosure will become more readily apparent and will be better understood by referring to the following detailed description of preferred embodiments, which are described hereinbelow with reference to the drawings wherein:

FIGS. 1-2 are perspective views of a cannula assembly and a seal assembly in accordance with the principles of the present disclosure;

FIG. 3 is a perspective view with parts separated of the cannula and seal assemblies of FIG. 1;

FIG. 4 is a perspective view of a seal mount depicted in FIG. 3;

FIG. 5A is cross-sectional view with the components of the seal mount depicted in FIG. 4 separated;

FIG. 5B is cross-sectional view of the seal mount depicted in FIG. 4;

FIG. 6 is a perspective view illustrating the seal mount depicted in FIG. 3 within a seal housing; and

FIG. 7 is a perspective view illustrating the cannula assembly and seal assembly accessing an, internal cavity with an instrument introduced therein.

### DETAILED DESCRIPTION OF PREFERRED EMBODIMENTS

The seal assembly of the present disclosure, either alone or in combination with a seal system internal to a cannula assembly, provides a substantial seal between a body cavity of a patient and the outside atmosphere before, during and after insertion of an instrument through the cannula assembly. Moreover, the seal assembly of the present invention is capable of accommodating instruments of varying diameters, e.g., from 5 mm to 15 mm, by providing a gas tight seal with each instrument when inserted. The flexibility of the present seal assembly greatly facilitates endoscopic surgery where a variety of instruments having differing diameters are often needed during a single surgical procedure.

The seal assembly contemplates the introduction and manipulation of various types of instrumentation adapted for insertion through a trocar and/or cannula assembly while maintaining a fluid tight interface about the instrumentation to preserve the atmospheric integrity of a surgical procedure from gas and/or fluid leakage. Specifically, the seal assembly accommodates off-axial insertion of the surgical instrument relative to the seal housing axis. This feature of the present disclosure desirably minimizes the entry and exit of gases and/or fluids to/from the body cavity. Examples of instrumentation include clip appliers, graspers, dissectors, retractors, staplers, laser probes, photographic devices, endoscopes and laparoscopes, tubes, and the like. Such instruments will be collectively referred to herein as “instruments or instrumentation”.

By virtue of its features, the seal assembly further defines a substantially reduced profile when assembled together and mounted to a cannula assembly. This reduced profile advantageously increases the working length of instruments introduced into the body cavity through the cannula assembly. In addition, the reduced profile permits enhanced angulation of a surgical instrument relative to the seal housing axis.

In the following description, as is traditional the term “proximal” or “leading” refers to the portion of the instrument closest to the operator while the term “distal” or “trailing” refers to the portion of the instrument remote from the operator.

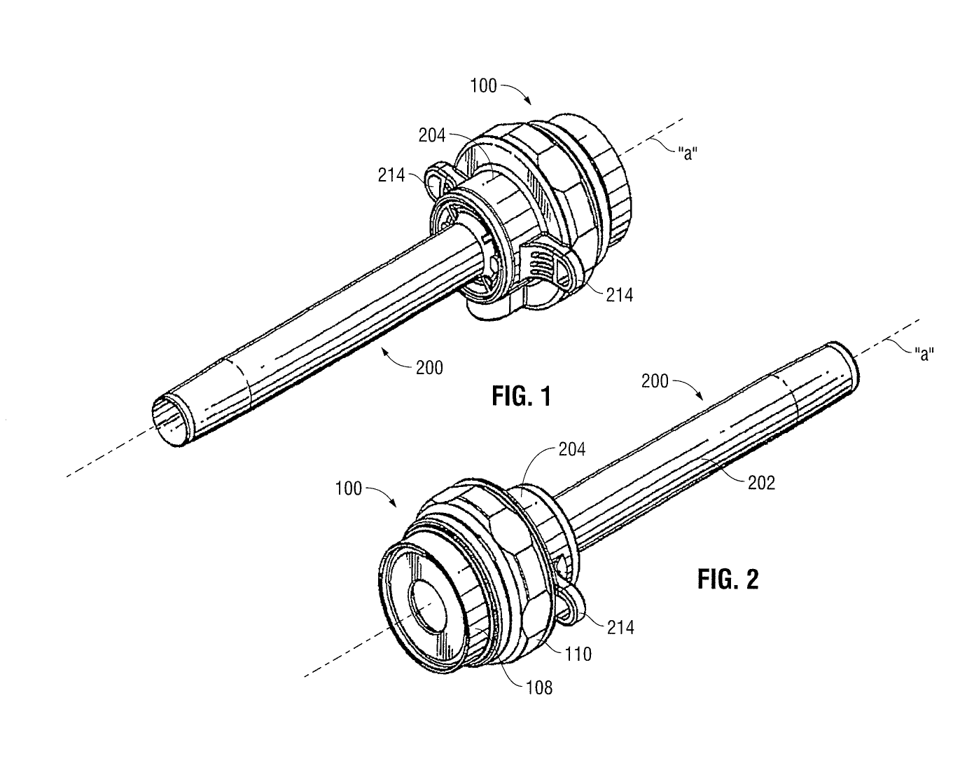

Referring now to the drawings, in which like reference numerals identify identical or substantially similar parts throughout the several views, FIGS. 1-2 illustrate the seal assembly 100 of the present disclosure mounted to cannula assembly 200. Cannula assembly 200 may be any conventional cannula suitable for the intended purpose of accessing a body cavity and permit introduction of instruments therethrough, such as the one described in commonly-owned United States Patent Publication No. 2005/0212221, the contents of which hereby incorporated in its entirety by reference. Cannula assembly 200 is particularly adapted for use in laparoscopic surgery where the peritoneal cavity is insufflated with a suitable gas, e.g., CO.sub.2, to raise the cavity wall from the internal organs therein. Cannula assembly 200 is typically used with an obturator assembly (not shown) which is a sharp pointed instrument positionable within the passageway of the cannula assembly 200. The obturator assembly is utilized to penetrate the abdominal wall and then subsequently removed from the cannula assembly to permit introduction of the surgical instrumentation utilized to perform the procedure.

Cannula assembly 200 includes cannula sleeve 202 and cannula housing 204 mounted to an end of the sleeve 202. Cannula sleeve 202 defines a longitudinal axis “a” extending along the length of sleeve 202. Sleeve 202 further defines an internal longitudinal passage dimensioned to permit passage of surgical instrumentation. Sleeve 202 may be formed of stainless steel or other rigid materials such as a polymeric material or the like.

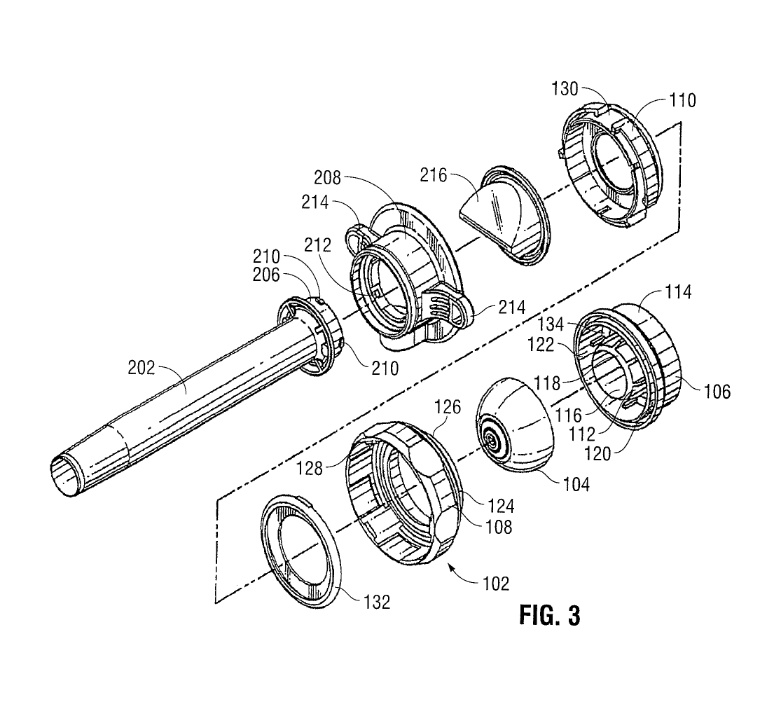

Cannula housing 204 includes two components, specifically, housing flange 206 which is attached to the proximal end of cannula sleeve 202 and main housing 208 as shown in FIG. 3. Main housing 208 is connectable to housing flange 206 by one or more suitable connection methods, such as, for example, through a bayonet coupling consisting of radially spaced tongues 210 on the exterior of housing flange 206 and corresponding recesses 212 within the interior of main housing 208. Main housing 208 further includes diametrically opposed housing grips 214 dimensioned and arranged for gripping engagement by the fingers of the user. Although shown and described as two components, cannula housing 204 may be a single component and attached to cannula sleeve 202 by any of the aforementioned means.

With reference to FIG. 3, in conjunction with FIGS. 1-2, cannula housing 204 further includes duck bill or zero closure valve 216 which tapers distally and inwardly to a sealed configuration as shown in the figure. Valve 216 is configured to function in a manner that is conventional in the art.

For a more detailed description of the cannula 200 and operative components associated therewith reference is again made to commonly-owned United States Patent Publication No. 2005/0212221.

Referring now to FIGS. 3-5B, in conjunction with FIGS. 1-2, seal assembly 100 will be discussed in detail. Seal assembly 100 includes seal housing, generally identified as reference numeral 102, and seal mount 104 which is disposed within the seal housing 102. Seal housing 102 houses the sealing components of the assembly and defines the outer valve or seal body of the seal assembly 100. Seal housing 102 defines central seal housing axis “b-b” which is preferably parallel to the axis “a-a” of cannula sleeve 202 and, more specifically, coincident with the axis “a-a” of the cannula. Seal housing 102 incorporates three housing components, namely, proximal, distal and inner housing components 106, 108, 110, respectively, which, when assembled together, form the seal housing 102. Assembly of housing components 106, 108, 110 may be affected by any of the aforementioned connection means discussed with respect to cannula housing 204. Further, seal housing 102 may be considered as having an upper housing portion 109 formed by components 106 108, as shown separately in FIG. 6, and a detachable lower housing portion formed by component 110.

Proximal housing component 106 defines inner guide wall 112 and outer wall 114 disposed radially outwardly of the inner guide wall 112. Inner guide wall 112 defines central passage 116 which is dimensioned to receive a surgical instrument “I” and laterally confine the instrument within seal housing 102. Inner guide wall 112 is generally cylindrical in configuration and terminates in a distal arcuate surface 118. Outer wall 114 defines first and second annular recesses 120, 122 adjacent its distal end. Recesses 120, 122 receive corresponding structure, e.g., annular lips 124, 126 of distal housing component 108 to facilitate connection of the two components.

Inner housing component 110 is disposed within the interior of distal housing component 108 and securely connectable to the distal housing component 108 through a bayonet coupling. Such coupling includes radially spaced tongues 128 which depend radially inwardly to be received within correspondingly arranged grooves or recesses 130 on the exterior of inner housing component 110. Coupling of distal and inner housing components 108, 110 is thereby affected through simple rotation of the components.

In the embodiment illustrated in FIG. 3, seal assembly 100 includes an optional skirt seal 132 mounted about the proximal end of inner housing component 110 or on the upper surface of the inner housing component (constituting a lower component) of the seal housing. Skirt seal 132 functions in minimizing the loss of insufflation gases through seal assembly 102. Skirt seal 132 also engages seal mount 104 and serves to bias the seal mount in a proximal direction against inner guide wall 112 of proximal housing 106 as will be discussed. Skirt seal 132 is preferably fabricated from a suitable elastomeric material or the like to provide a spring-like characteristic sufficient to appropriately bias seal mount 104.

In embodiments, seal mount 104 may be accommodated within an annular space 134 defined between inner and outer walls 112, 114 of proximal housing component 106. In certain embodiments, seal mount 104 may be mounted in a manner which permits angulation of the seal mount 104 relative to seal axis “b-b”.

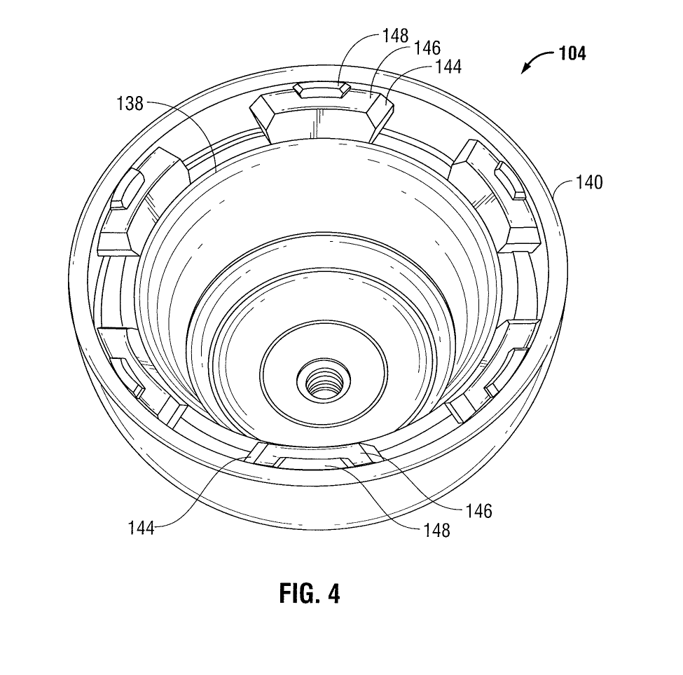

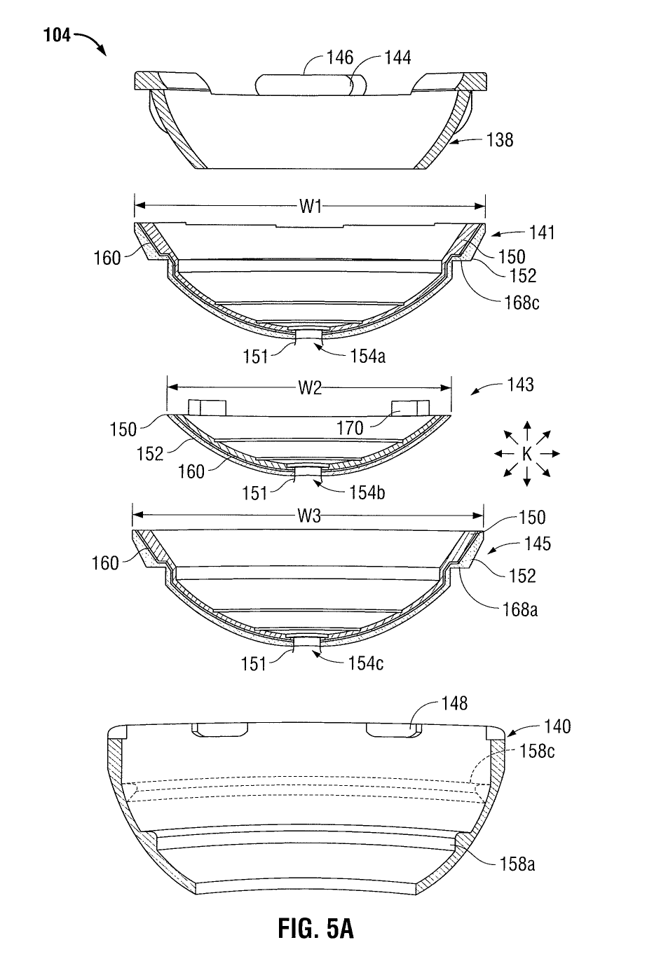

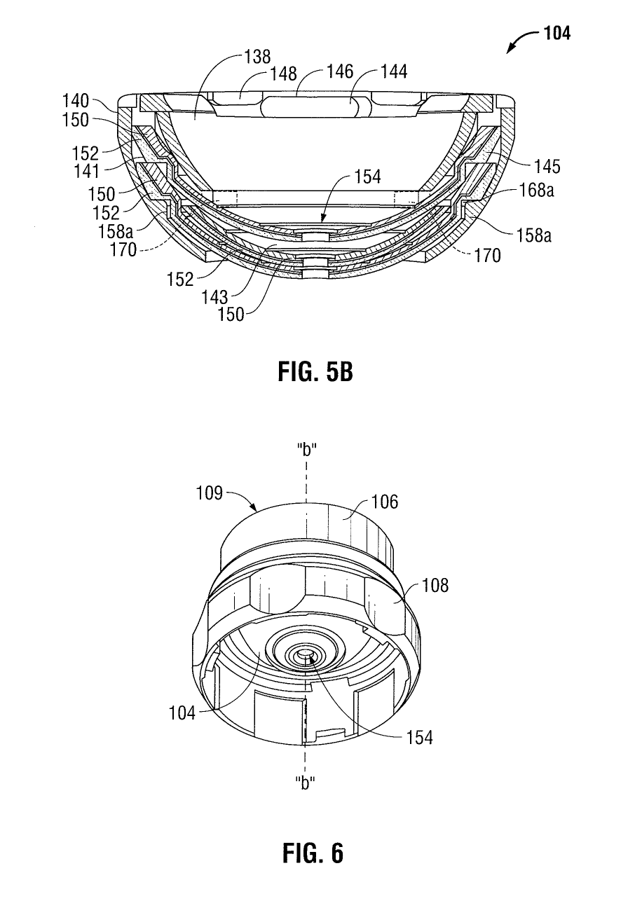

Referring now to FIGS. 4-5B, in conjunction with FIG. 3, the components of seal mount 104 will be discussed in further detail. Seal mount 104 includes first and second seal housings 138, 140 and a plurality of resilient seal members 141, 143, 145 mounted between the housings 138, 140. In the illustrated embodiment seal members 141, 143, 145 include three (3) resilient seal members, a first seal member 141, an second seal member 143 and a third seal member 145. First and second seal housings 138, 140 and seal members 141, 143 and 145 each define a generally spherical configuration, e.g., a portion of a spherical or hemispherical configuration, as shown. First seal housing 138 is preferably seated within second seal housing 140 thereby at least partially enclosing seal member 141, 143 and 145. First seal housing 138 may include a plurality of mounting legs 144 radially spaced about the outer periphery of the seal housing component 138. Legs 144 define locking surfaces 146 which extend in general transverse relation to the axis “b-b” of seal assembly 200. Similarly, second seal housing 140 includes a plurality of corresponding locking detents 148 spaced about the interior of the housing 140. During assembly, the first, second and third seal members 141, 143 and 145, respectively, are positioned within second seal housing 140. First seal housing 138 is arranged above the second seal housing whereby mounting legs 144 of the first seal housing 138 are longitudinally aligned with the spaces defined between detents 148 of second seal housing 140. First seal housing 138 is advanced within second seal housing 140 and the housings 138, 140 are rotated relative to each other whereby mouthing legs 144 are positioned beneath detents 148 thereby coupling the first and second seal housing 138 and 140. Other means for coupling first and second seal housings 138, 140 are also envisioned such as, for example, with mechanical arrangements and/or adhesives and cements.

Second seal housing 140 may include one annular internal ledge 158a extending along at least a portion of the inner periphery and, in some embodiments, may include a second annular internal ledge 158c (shown in phantom). Ledges 158a and 158c assist in securing seal members 141, 143, 145 within seal housings 138, 140 as will be discussed.

First and third seal members 141, 145 each define an outer periphery with a respective substantially planar annular ledge 168a and 168c. In one embodiment, annular ledge 168a of third seal member 145 rests on annular internal ledge 158c of second seal housing 140 and annular ledge 168a rests on outer periphery of third seal member 145 in the assembled condition of seal mount 104 (FIG. 5B). In another embodiment, instead of residing on outer periphery of third seal member 145, annular internal ledge 168c of first seal may reside on second annular internal ledge 158c of the second seal housing 140 in the assembled condition of the components. Regardless of the mounting methodology, first and third seal members 141 and 145, respectively, are mounted in fixed relation relative to seal mount 104 such that the first and third seal members 141 and 145, respectively, will not angulate during corresponding movement of the surgical instrument “I”. The term “angulate” at least includes pivoting movement of the first and third seals relative to the central longitudinal axis. However, it is envisioned that first and third seal members, 141 and 145, respectively, may rotate about the central longitudinal axis “a-a.”

Second seal 143 is mounted between first and third seal members 141 and 145 and defines a width “w2” across the second seal member 143 which is less than the corresponding widths “w1, w3” of first and third seal members 141, 145. The radii of curvature of the first, second and third seal members 141-145 may generally approximate each other.

Second seal member 143 is devoid of an annular ledge. Second seal member 143 may angulate (e.g., move or slide in the direction of directional arrows “k” and may rotate about the central longitudinal axis) with respect to the longitudinal axis “b-b” within seal mount 104 and relative to first and third seal members 141 and 143. A lubricous coating on the proximal and distal faces of the second seal member 143 may facilitate the angular movement of the second seal member 143.

Thus, during angular movement of the surgical instrument “I”, a substantial sealed relation is maintained about the surgical instrument “I” through the cooperative angular movement of the second seal member 143 even in the presence of any gapping or “cat-eyeing” occurring between the instrument and the first and third seal members 141 and 145.

Second seal member 143 may also include a plurality of radially spaced projecting elements 170 projecting in a general longitudinal direction from the periphery of the second seal member 143 toward first seal member 141. Projecting elements 170 may comprise an elastomeric deformable material which deforms during angulation of the second seal member 143, but will normally bias the second seal member 143 toward a position where a seal aperture 154 is generally aligned with the central longitudinal axis “a-a”. Projecting elements 170 may engage the underside of a peripheral edge of first seal member 141 during angulation of the second seal member 143. The fixed characteristic of the peripheral edge of first seal member 141 will apply a counterforce to the projecting elements 170 whereby the second seal member 143 is normally biased toward an adjacent position.

Projecting elements 170 may also engage an outer or lower surface of first seal member 141 to maintain a spaced relation between the first and second seal members 141 and 143, respectively. The spaced relation may facilitate angulation of the second seal member 143 relative to the first seal member 141.

Seal members 141, 143 and 145 of seal mount 104 are secured in interposed relation between first and second seal housings 138, 140. Each of the seal members 141, 143 and 145 may include a resilient layer or center material 160 (e.g., polyisoprene or natural rubber) with first and second layers of fabric 150,152 impregnated on the respective proximal and distal surfaces of the resilient center material 160. Fabric layers 150 and 152 may be formed of any suitable fabric for example, a SPANDEX material containing about 20% LYCRA and about 80% NYLON available from Milliken. In embodiments, the first, second and third seal members 141, 143 and 145, respectively, include resilient material and fabric material which resists deformation of the respective apertures 154, as well as tearing of the seal members 141, 143 and 145. A suitable seal member or seal type is disclosed in commonly assigned U.S. patent application Ser. No. 09/449,368, filed Nov. 24, 1999, the contents of which are incorporated herein by reference. A central aperture 154 for sealed reception of a surgical instrument “I” is defined by three central apertures 154a-154c defined by a corresponding seal member 141, 143 and 145 (as best shown in FIG. 4). Each of the seal members 141, 143 and 145 (e.g., seal member 141) is arranged such that first the layer 150 extends or overlaps into a corresponding aperture, e.g., aperture 154a (FIG. 5A). In this manner, the fabric (which is stronger relative to the resilient material) is positioned to engage the surgical instrument “I” upon passage through aperture 154 of seal members 141, 143 and 145 thereby protecting the resilient material defining the aperture 154. This advantageously minimizes the potential of piercing, penetrating or tearing of the resilient material by the instrument. Alternatively, an additional layer of fabric 151 on the proximal surface of seal member 141 may be superposed and arranged to drape within aperture 154.

Although each of seal members 141, 143 and 145 is disclosed as an impregnated fabric arrangement, it is appreciated that other seal types may be used and still achieve the objectives of the present disclosure.

Seal assembly 100 may be associated with, or joined to, cannula assembly 200 in a variety of ways. In a preferred embodiment, seal housing 102 of seal assembly 100 and cannula housing 204 of cannula assembly 200 are adapted to detachably engage each other, e.g., through a bayonet lock or like mechanical means. As previously discussed, proximal and distal housing components 106, 108 may define an upper housing component 109 (FIG. 6) which is mountable directly to cannula assembly 200. Alternatively, inner housing portion 110 which defines a lower housing component may be directly mounted to cannula assembly 200 independent of the upper housing component 109. Specifically, the lower housing component 110 which houses seal mount 104 may be mounted to cannula assembly independent of the remaining housing components. The upper housing may then be mounted to lower housing or cannula assembly 200 as needed. Even further, upper housing component 109 may be mounted to cannula assembly 200 without lower housing component 110. Other means of joining seal assembly 100 to cannula assembly 200 will be readily apparent to one of ordinary skill in the art.

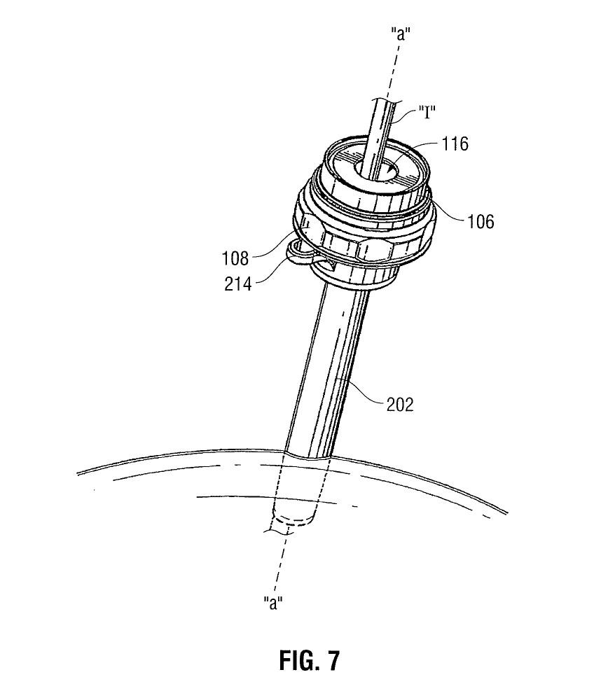

Referring now to FIG. 7, use of the seal assembly 100 and cannula assembly 200 in connection with introduction of a surgical instrument will be discussed. Seal assembly 100 is mounted to cannula assembly 200 which is previously introduced into an insufflated abdominal cavity. An instrument “I” is inserted into seal assembly 100 through passage 116 of inner cylindrical guide wall 112 in seal housing 102. If the axis of the instrument is not perfectly aligned (i.e., off-axial insertion of the instrument) with the axis “a-a” of cannula assembly 200 or axis “b-b” of seal assembly 100, then the surgical instrument “I” will contact the inner guide wall 112 and/or the inner surface of first seal member 141. Contact with the first seal member 141 can cause some deformation of the first seal member 141. The instrument “I” may slide along the surface of the seal mount 104 and/or the first seal member 141, to the aperture 154. Second seal member 143 stretches to accommodate a diameter of surgical instrument “I”, as necessary. The surgical instrument “I” passes further distally into the cannula housing 204 passing through duckbill valve 216 and cannula sleeve 202 into the body cavity. Once the surgical instrument “I” is disposed within the aperture 154, the second seal member 143 is free to move or “float” with respect to the first and third seal members 141, 145, respectively, and the seal housing 102, while allowing the first and third seal members 141 and 145, respectively, and second seal member 143 to maintain sealing engagement with the instrument passed therethrough, as well as maintaining the seal around the seal mount 104.

While the invention has been particularly shown, and described with reference to the preferred embodiments, it will be understood by those skilled in the art that various modifications and changes in form and detail may be made therein without departing from the scope and spirit of the invention. Accordingly, modifications such as those suggested above, but not limited thereto, are to be considered within the scope of the invention.

What is claimed is:

1. A surgical portal assembly, which comprises:
a portal housing;
a portal member mounted to the portal housing, the portal member dimensioned for positioning within tissue to access an underlying surgical site, the portal housing and the portal member defining a central longitudinal axis and a longitudinal passageway for passage of a surgical object; and
a seal mount mounted to the portal housing, the seal mount including first and second seal members, each of the first and second seal members having inner surfaces defining a passage for reception of the surgical object in substantial sealed reception, the first seal member adapted for angulating movement relative to the central longitudinal axis upon angulation of the surgical instrument while substantially maintaining the sealed reception of the surgical object, the second seal member being generally fixed from angulating movement.

2. The portal assembly according to claim 1 including a third seal member, the third seal member being generally fixed from angulating movement.

3. The portal assembly according to claim 2 wherein the first seal member is disposed between the second and third seal members.

4. The portal assembly according to claim 3 wherein each of the first, second and third seal members define a general spherical segment.

5. The portal assembly according to claim 4 wherein the second seal member is in contacting relation with at least one of the first and third seal members.

6. The portal assembly according to claim 1 wherein the inner surfaces of the first seal member define an open aperture in the absence of the surgical object.

7. The portal assembly according to claim 1 wherein the first seal member is normally biased to an aligned position where the seal passage of the first seal member is in general longitudinal alignment with the central longitudinal axis.

8. The portal assembly according to claim 7 wherein the first seal member includes resilient biasing segments extending in a general proximal direction and dimensioned to operatively engage the portal housing, the biasing segments adapted to bias the seal passage to the aligned position.
